(19) United States
(12) Patent Application Publication
(10) Pub. No.: US 2004/0197911 A1
(43) Pub. Date: Oct. 7, 2004

(54) Novel 88 phage vectors

(76) Inventors: Katherine S. Bowdish, Del Mar, CA (US); Shana Frederickson, Solana Beach, CA (US); Martha Wild, Solana Beach, CA (US)

Correspondence Address: Mark Farber, c/o, Alexion Pharmaceuticals, Inc., 352 Knotter Drive, Cheshire, CT, 06410, US

(21) Appl. No.: 10/134,199

(22) Filed: Apr. 26, 2002

Related U.S. Application Data
(60) Provisional application No. 60/287,239, filed on Apr. 27, 2001.

(51) Int. Cl. C12N 15/86

(52) U.S. Cl. 435/456; 435/320.1

(57) ABSTRACT

A phage genome is engineered to include a novel restriction site at one of two different positions. In a first embodiment, a restriction site is inserted into the phage genome between the end of gene IV and the MOS hairpin which serves as a phage packaging signal for newly synthesized single strands of phage DNA. In a second embodiment, a restriction site is inserted into the phage genome after the MOS hairpin and prior to the minus strand origin. Once the phage genome is modified to contain the new restriction site, the vector can be engineered to be a “88” vector by inserting at the new restriction site a nucleotide sequence encoding at least a functional domain of pVIII and at least a first cloning site for receiving a gene encoding a polypeptide to be displayed and, optionally a second cloning site for receiving a second gene encoding a polypeptide capable of dimerizing with the polypeptide to be displayed. In particularly useful embodiments, the novel vectors are engineered to produce phage particles that display antibodies.

### BACKGROUND

1. Technical Field

This disclosure relates to phage vectors useful for generating phage display libraries. More specifically this disclosure relates to vectors useful for display of antibodies on phage particles.

2. Background of Related Art

Filamentous bacteriophage consist of a circular, single-stranded DNA molecule surrounded by a cylinder of coat proteins. There are about 2,700 molecules of the major coat protein pVIII that envelope the phage. At one end of the phage particle, there are five copies each of gene III and VI proteins (pIII and pVI) that are involved in host-cell binding and in the termination of the assembly process. The other end contains five copies each of pVII and pIX that are required for the initiation of assembly and for maintenance of virion stability.

In recent years, vectors have been developed that allow the display of foreign peptides on the surface of a filamentous phage particle. By insertion of specific oligonucleotides or entire protein coding regions into genes encoding specific phage capsid proteins, chimeric proteins can be produced which are able to be assembled into phage particles. This results in the display of the foreign protein or peptide on the surface of the phage particle.

The display of peptides and proteins on the surface of bacteriophage represents a powerful methodology for selection of rare members in a complex library and for carrying out molecular evolution in the laboratory. The ability to construct libraries of enormous molecular diversity and to select for molecules with predetermined properties has made this technology applicable to a wide range of problems.

A few of the many applications of such technology are: i) phage display of natural peptides including, mapping epitopes of monoclonal and polyclonal antibodies and generating immunogens; ii) phage display of random peptides, including mapping epitopes of monoclonal and polyclonal antibodies, identifying peptide ligands, and mapping substrate sites for proteases and kinases; and iii) phage display of protein and protein domains, including directed evolution of proteins, isolation of antibodies, and cDNA expression screening.

One important application of phage display has been to construct combinatorial peptide libraries. Synthetic oligonucleotides, fixed in length but with unspecified codons, can be cloned as fusions to genes III or VIII of phage where they are expressed as a plurality of peptide:capsid fusion proteins. The libraries, often referred to as random peptide libraries, can then be tested for binding to target molecules of interest. This is most often done using a form of affinity selection known as “biopanning” or simply “panning”.

A variety of commonly used display vectors, with their name, site of expression, restriction site used, marker carried on the vector, and reference, are provided in Phage Display of Peptides and Proteins, A Laboratory Manual, ed. Kay et al., Academic Press, 1996, page 38 and reproduced in the following table:

| Vector | Gene | Rest. Site(s) | Marker |
|---|---|---|---|
| fUSE5 | III | BglI-S-BglI | tet^{R} |
| fAFF1 | III | BstXI-S-BstXI | tet^{R} |
| fd-CAT1 | III | PstI-S-XhoI | tet^{R} |
| M663 | III | XhoI-S-XbaI | lacZ⁺ |
| fdtetDOG | III | ApaLI-S-NotI | tetR |
| 33 | III | SfiI-S-NotI | |
| 88 | VIII | | |
| Phagemid | III | | amp^{R} |
| pHEN1 | III | SfiI-S-NotI | amp^{R} |
| pComb3 | III | | |
| pComb8 | VIII | | |
| pCANTAB 5E | III | SfiI-SNotI | amp^{R} |
| p8V5 | VIII | BstXI-S-BstXI | amp^{R} |
| λSurfZap | III | NotI-S-SpeI | amp^{R} |

A variety of phage and phagemid vectors have been constructed and utilized for phage display. Each of the existing vectors has its advantages and disadvantages. By convention, vectors that fuse a gene of interest whose protein product is to be displayed to gene VIII have been categorized as either type 8, type 8+8 or type 88. Type 8 vectors are phage vectors where all copies of gene VIII are fused to a gene of interest for display. With approximately 2700 copies of pVIII on the surface of the phage particle, there is little tolerance for large inserts to be displayed on the phage surface. In addition, strong avidity effects due to multivalent display reduce selective pressure for high affinity that is commonly desired and may be taken into account.

The 8+8 vectors are phagemid vectors. In the phagemid system, helper phage are required to package the phagemid genome into a phagemid particle that is extruded out of the cell. In 8+8 vectors, the gene of interest is fused to a copy of gene VIII on the plasmid, while the helper phage retains a wildtype, unfused copy of gene VIII. Hence, the coat of the phagemid particle is made up of both wildtype and pVIII fusion proteins leading to more stability and a loss of some avidity effects. However, since both helper phage and phagemid particles are produced from the same cell, both helper phage and phagemid viral particles will have fusion proteins on the surface leading to a loss of the corresponding genetic information from helper phage particles that inadvertently display selected proteins.

The type 88 vectors are phage vectors where both a fused and unfused copy of gene VIII are present on the phage vector. The phage vector system is less complex in that helper phage are not required. Additionally, there is no loss of selected clones that result from inadvertent display on the helper phage surface. However, the presently known 88 vectors are derivatives of fd-tet, where an insert conferring tetracycline resistance was introduced at a convenient restriction site. Unfortunately, the insert disrupts the minus strand origin of replication, leading to a defect in minus strand synthesis. As a result, these vectors have a very low intracellular RF copy number, making vector production for cloning as well as library amplification difficult. In addition, the size of the insert conferring tetracycline resistance is approximately 2.6 kb. This large insert, in addition to insertions into the phage for protein of interest display (including promoter, ribosomal binding sites, signal sequences, stuffer fragments in the case of the cloning vectors, and antibody genes in the case of antibody display) yield a large phage genome that is not packaged as efficiently as smaller phage genomes. The fd-tet vector has served as the starting point of construction of a variety of phage vectors including the fuSE vectors of G. Smith (Scott and Smith, Science, Vol. 249, pages 386-390, 1990), fd-CAT1 (McCafferty et al., Nature (London), Vol. 348, pages 552-554, 1990) and fdtetDOG of Hoogenboom et al., Nucleic Acid Res., Vol. 19, pages 4133-4137, 1991.

### SUMMARY

This disclosure describes novel phage vectors useful for generating phage display libraries. The novel vectors described herein are produced as the result of modification of a phage genome at an artificially created cloning site not employed in previous phage vector constructions.

Specifically, a phage genome is engineered in accordance with this disclosure to include a restriction site at one of two different positions. In a first embodiment, a restriction site is inserted into the phage genome between the end of gene IV and the MOS hairpin which serves as a phage packaging signal for newly synthesized single strands of phage DNA. In a second embodiment, a restriction site is inserted into the phage genome after the MOS hairpin and prior to the minus strand origin.

Once the phage genome is modified to contain the new restriction site, cloning sites for receiving one or more genes can be inserted into the phage vector in accordance with this disclosure. Preferably, the vector is engineered to be a “88” vector by inserting at the new restriction site a nucleotide sequence encoding at least a functional domain of pVIII and at least a first cloning site for receiving a gene encoding a polypeptide to be displayed. In an alternative embodiment, the 88 vector is engineered to cause display of a dimeric (e.g., heterodimeric) species by inserting first cloning site for receiving first gene encoding a polypeptide to be displayed and a second cloning site for receiving a second gene encoding a polypeptide capable of dimerizing with the polypeptide to be displayed, thereby resulting in display of a dimeric polypeptide or protein. The first and second cloning sites, if desired and practical, can be inserted with the nucleotide sequence encoding at least a functional domain of pVIII as part of a single cassette referred to herein as a display cassette.

In particularly useful embodiments, the novel vectors are engineered to produce phage particles that display antibodies. After creation of the novel restriction site and insertion of the display cassette within a phage genome, a first gene encoding an antibody heavy chain Fd is inserted adjacent the nucleotide sequence encoding at least a functional domain of pVIII to produce a pVIII fused with a heavy chain Fd. A second gene encoding an antibody light chain is also inserted into the vector.

### BRIEF DESCRIPTION OF THE DRAWINGS

FIG. 1 is a flow chart illustrating the strategy for making a vector based on modification of the f1 genome between gene IV and the MOS hair pin;

FIG. 2 is a flow chart illustrating the strategy for making a vector based on modification of the f1 genome between the MOS hairpin and the minus strand origin;

FIG. 3 is a map of the vector produced in Example 1;

FIG. 4a is the sequence (Seq. ID No. 2) of cassette 1a used in Example 1;

FIG. 4b is the sequence (Seq. ID No. 7) of cassette 2 used in Examples 1 and 2;

FIG. 4c is the sequence (Seq. ID No. 12) of cassette 3 used in Examples 1 and 2;

FIG. 4d is the sequence (Seq. ID No. 22) of an alternative display cassette useful in making an 88 vector in accordance with this disclosure;

FIG. 5a is a map of the pAX131 vector;

FIGS. 5b-e show the sequence (Seq. ID No.13) of the pAX131 vector

FIG. 6 is the sequence (Seq. ID No. 14) of the synthetic gVIII portion of cassette 3.

FIG. 7 shows the alignment of the oligos for preparation of the synthetic gVIII; and

FIG. 8 shows a map of the vector pAX131-gVIII;

FIG. 9 is the sequence (Seq. ID No. 23) of the final inserted construct resulting from the insertion of cassettes 1a, 2 and 3 in Example 1;

FIG. 10 is a map of the vector produced in Example 2;

FIG. 11 is the sequence (Seq. ID No. 25) for cassette 1b used in Example 2; and

FIG. 12 is the sequence (Seq. ID No. 30) of the final construct resulting from the insertion of cassettes 1b, 2 and 3 as described in Example 2.

### DETAILED DESCRIPTION OF PREFERRED EMBODIMENTS

The novel vectors described herein are prepared by modifying a phage genome. While the following description is provided with respect to the f1 genome as the starting material, it should be understood that other phage genomes (e.g., M13, fd, etc.) can be used as the starting material. Additionally, when the following description refers to “pVIII”, it should be understood that either full pVIII or a truncated version or fragment thereof is contemplated (unless the context indicates otherwise) provided the display function of the protein is maintained.

In one embodiment, the present vectors are the result of modification of the f1 genome between gene IV and the hairpin which serves as a packaging signal (MOS). First, the phage genome is engineered to contain a novel restriction site at this location. Then at least a first cloning site and a nucleotide sequence encoding at least a functional domain of pVIII are inserted at the newly formed restriction site. A first gene encoding a polypeptide to be displayed can be inserted at the first cloning site. Because the first cloning site is adjacent the nucleotide sequence encoding at least a functional domain of pVIII, once the first gene is inserted, the vector effectively encodes a fusion protein of pVIII and a polypeptide to be displayed by the phage particle. Any polypeptide that can be displayed by phage can be fused to pVIII. Non-limiting examples of polypeptides that can be displayed include naturally occurring and synthetic enzymes, hormones, antibodies, antigens, toxins and cytokines. For a nonlimiting list of proteins and protein domains that can be displayed, see Phage Display of Peptides and Proteins, A Laboratory Manual, Kay et al., ed., Academic Press, 1996.

Optionally, a second cloning site is also inserted at the novel restriction site. The second cloning site is adapted to receive a second gene that encodes a polypeptide that can dimerize with the polypeptide fused to pVIII. In this manner, display of a dimeric species (e.g., a heterodimeric species) can be achieved. Where monomeric display of a single polypeptide or protein is intended, the second gene can be eliminated.

In a particularly preferred embodiment, the polypeptide fused to pVIII is an antibody heavy chain Fd and the modification to the f1 genome also involves inserting a site for cloning into the vector a second gene encoding an antibody light chain. In this manner, the vector can be used to make phage particles that display antibody libraries.

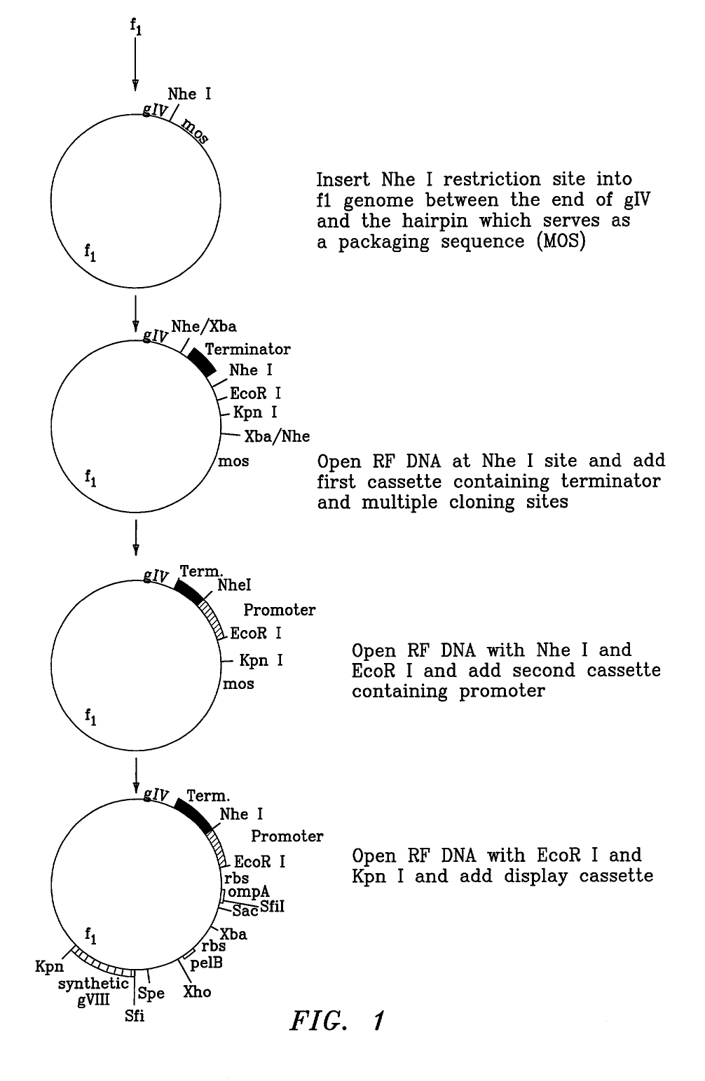

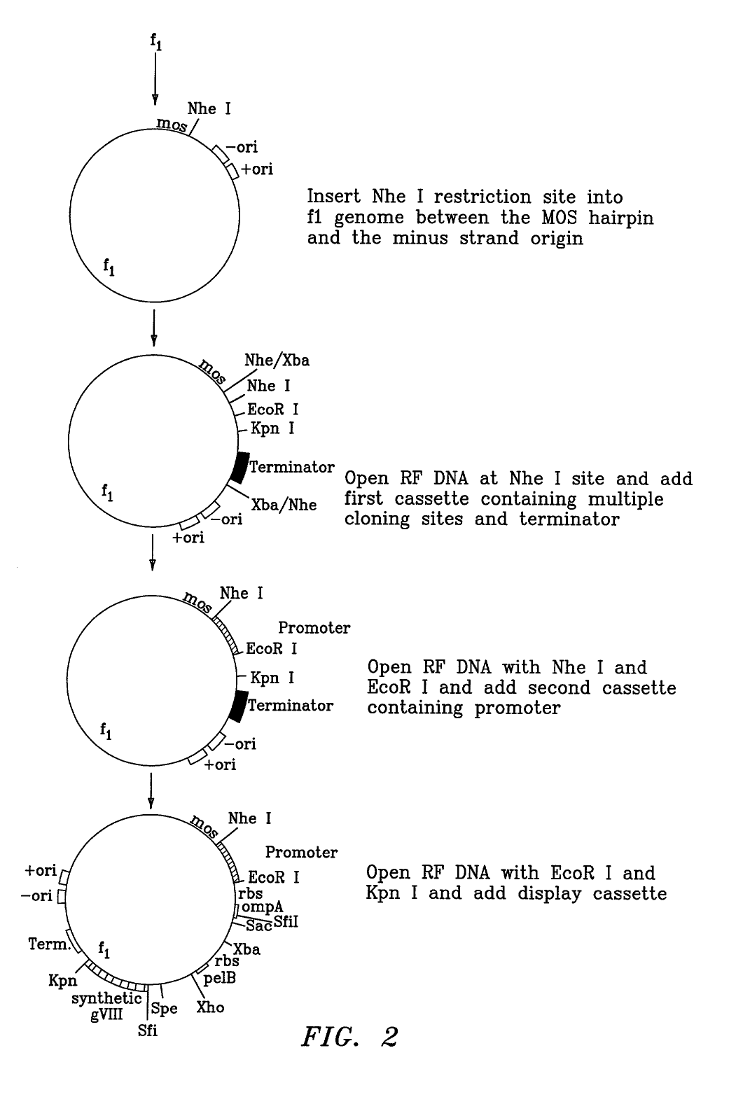

FIG. 1 is a flow chart showing the steps involved in a particularly useful method for producing a phage vector capable of generating phage display of polypeptides (e.g., libraries of antibodies) in accordance with this disclosure. In the first step, a restriction site is introduced into the into the f1 genome between the end of gene IV and the hairpin which serves as a packaging signal (MOS). The restriction site can be any known restriction site. Suitable restriction sites for insertion include, but are not limited to Nhe I, Hind III, Nco I, Xma I, Bgl II, Bst I, Pvu I, etc. It should be understood that if a restriction site selected for insertion is present in the native genome, it may be desirable to remove or disable the native restriction site to avoid unwanted digestion during further processing. The restriction site can be inserted using any technique known to those skilled in the art. In a particularly useful embodiment, overlap PCR is used to generate a restriction fragment containing the desired restriction site. This fragment is then cloned into the phage genome at suitable sites.

In the next step, the replicative form (RF) DNA is opened by digestion and a first cassette containing a terminator and multiple cloning sites is added. Depending on the particular restriction site inserted in the first step, specific methods for opening the RF DNA are known to and readily selected by those skilled in the art. Preferably the first cassette is engineered to include overhangs which, when combined with the ends of the DNA formed by the digestive opening thereof at the inserted restriction site will create a hybrid site that will no longer be recognized as the inserted restriction site. In this manner, subsequent cloning steps advantageously occur at the cloning sites within the first cassette. If desired, one of the cloning sites within the first cassette can be the same as the restriction site inserted in the first step to decrease the number of different enzymes employed in the process.

Methods of preparing suitable cassettes for this and subsequent steps are within the purview of those skilled in the art. For example, suitable cassettes can be created using overlapping oligonucleotides (“oligos”) in a PCR fill in reaction. As another example, cassettes can be created using long complementary oligos which can form a double stranded DNA cassette. The oligos are mixed in a 1:1 ratio, heat denatured and slowly cooled to allow the duplexed cassette to form. Other suitable techniques for creating cassettes will be evident to those skilled in the art.

Next, the process shown in FIG. 1 involves again opening the RF DNA at one of the cloning sites within the first cassette and inserting a second cassette that includes a promoter. Any promoter recognized by a host cell can be employed. Suitable promoters include, but are not limited to, ara, lac and trc promoters. The promoter drives expression of other sequences inserted into the vector, such as, for example expression of the pVIII fusion protein and any polypeptides intended to dimerize therewith.

After insertion of the second cassette, the RF DNA is again opened at one of the other cloning sites contained in the first cassette, and a display cassette is added. As noted above, the display cassette contains at least a nucleotide sequence encoding at least a functional domain of pVIII and a first cloning site adapted to receive a gene encoding a polypeptide to be displayed. The nucleotide sequence encoding at least a functional domain of pVIII can be natural or synthetic. Preferably, the display cassette contains a synthetic gene VIII to avoid having identical native gene VIII sequences at two different locations within the vector. The nucleotide sequence can encode a truncated pVIII provided the display function of the protein is maintained.

The display cassette contains at least a first cloning site for receiving a first gene encoding a polypeptide to be displayed. The cloning site is a region of the nucleic acid between two restriction sites, typically with a nonessential region of nucleotide sequence (commonly referred to as a “stuffer” sequence) positioned therebetween. In the flow chart of FIG. 1, the first cloning site is defined by XhoI and SpeI restriction sites adjacent to the synthetic gene VIII. As those skilled in the art will appreciate, a suppressible stop codon could be positioned between the first gene and the nucleotide sequence encoding at least a functional domain of pVIII such that fusion display is obtained in a suppressing host (as long as the first gene is inserted in-frame) and a secreted protein without pVIII is obtained in a non-suppressing host.

The display cassette optionally also contains a second cloning region for receiving a second gene encoding a polypeptide that can dimerize with the polypeptide to be displayed. For example, where the vector expresses a heavy chain Fd fused to pVIII, the second gene preferably encodes an antibody light chain. As with the first cloning site, the second cloning site is a region of the vector between two restriction sites, typically with a stuffer positioned therebetween. In the flow chart of FIG. 1, the second cloning site is defined by SacI and XbaI restriction sites. It should of course be understood that where a polypeptide other than an antibody is to be displayed (such as, for example, where monomeric display of a single polypeptide or protein is intended) a second gene need not be cloned into the vector. In such cases the second cloning site can either remain unused, or eliminated entirely. As those skilled in the art will also appreciate, where a single chain antibody is encoded by the first gene, there is no need to insert a second gene into the vector at the second cloning site.

Thus, the phage vector produced by the process illustrated in FIG. 1 will be a modified f1 genome that contains, after the native gene IV but before the MOS hairpin, a terminator, a promoter, a cloning region for receiving a gene encoding an antibody light chain, a cloning region for receiving a gene encoding an antibody heavy chain Fd to be displayed and a synthetic gene VIII.

In another embodiment, the present vectors are the result of modification of the f1 genome between the hairpin which serves as a packaging signal (MOS) and the minus strand origin. After engineering a novel restriction site at this location, the vector has inserted at this site at least a nucleotide sequence encoding a pVIII and a cloning site for receiving a first gene encoding a polypeptide to be fused to pVIII and thus displayed by the phage particle. Suitable polypeptides to be displayed are those described above in connection with the previous embodiment. Optionally, a cloning site for receiving a second gene is also inserted at this site. The second gene preferably encodes a polypeptide that can dimerize with the polypeptide fused to pVIII. In this manner, display of a dimeric species (e.g., a heterodimeric species) can be achieved. Where monomeric display of a single polypeptide or protein is intended, the second gene can be eliminated.

In a particularly preferred embodiment, the polypeptide fused to pVIII is a heavy chain Fd and the modification to the f1 genome also involves inserting a site for cloning into the vector a second gene encoding an antibody light chain. In this manner, the vector can be used to make phage particles that display antibody libraries.

An example of a method of this alternative embodiment of forming a phage vector for generating phage display libraries of antibodies in accordance with this disclosure is shown in the flow chart of FIG. 2. In this embodiment, the first step involves introducing a restriction site into the f1 genome between the hairpin which serves as a packaging signal (MOS) and the minus strand origin. The restriction site can be any known restriction site. Suitable restriction sites for insertion include Nhe I, Hind III, Nco I, Xma I, Bgl II, Bst I, Pvu I, etc. It should be understood that if a restriction site selected for insertion is present in the native genome, it may be desirable to remove or disable the native restriction site to avoid unwanted digestion during further processing. The restriction site can be inserted using any technique known to those skilled in the art. In a particularly useful embodiment, overlap PCR is used to generate a restriction fragment containing the desired restriction site. This fragment is then cloned into the phage genome at suitable sites.

In the next step, the replicative form (RF) DNA is opened by digestion and a first cassette containing multiple cloning sites and a terminator is added. Depending on the particular restriction site inserted in the first step, specific methods for opening the RF DNA will be known to and readily selected by those skilled in the art. Preferably the first cassette is engineered to include overhangs which, when ligated with the ends of the DNA formed by digestion at the inserted restriction site will create a hybrid site that will no longer be recognized as the inserted restriction site. In this manner, subsequent cloning steps advantageously occur at the cloning sites within the first cassette. If desired, one of the cloning sites within the first cassette can be the same as the restriction site inserted in the first step to decrease the number of different enzymes employed in the process.

Next, the process shown in FIG. 2 involves again opening the RF DNA at one of the cloning sites within the first cassette and inserting a second cassette that includes a promoter. Any promoter recognized by the host cell can be employed. Suitable promoters include, but are not limited to, ara, lac and trc promoters.

After insertion of the second cassette, the RF DNA is again opened at one of the other cloning sites contained in the first cassette, and a display cassette is added. As shown in the flow chart of FIG. 2, the display cassette contains a synthetic gVIII and at least a first cloning site for receiving a first gene that encodes a polypeptide to be displayed, such as, for example, an antibody heavy chain Fd. The display cassette optionally contains a second cloning region for receiving a second gene, such as, for example, a gene encoding an antibody light chain. It should of course be understood that where a polypeptide other than an antibody is to be displayed (such as, for example, where monomeric display of a single polypeptide or protein or display of a single chain antibody is intended) a second gene need not be cloned into the vector.

Thus, the phage vector produced by the process illustrated in FIG. 2 will be a modified f1 genome that contains, after the MOS hairpin but before the minus strand origin, a promoter, an antibody cloning region for receiving a gene encoding an antibody light chain, an antibody cloning region for receiving a gene encoding an antibody heavy chain Fd to be displayed, a synthetic gene VIII and a terminator.

Optionally, a selectable marker can be added to the present vectors. Non-limiting examples of suitable markers include tetracycline or kanamycin resistance. There are multiple positions within the phage genome where a selectable marker could be inserted provided that transcriptional control elements are recreated if necessary so that phage particles can still be produced.

The vectors described herein can be transformed into a host cell using known techniques (e.g., electroporation) and amplified. The vectors described herein can also be digested and have a first gene and optionally a second gene ligated therein in accordance with this disclosure. The vector so engineered can be transformed into a host cell using known techniques and amplified or to effect expression of polypeptides and/or proteins encoded thereby to produce phage particles displaying single polypeptides or dimeric species. Those skilled in the art will readily envision other uses for the novel vectors described herein. The following examples illustrate the present invention without limiting its scope.

### EXAMPLE I

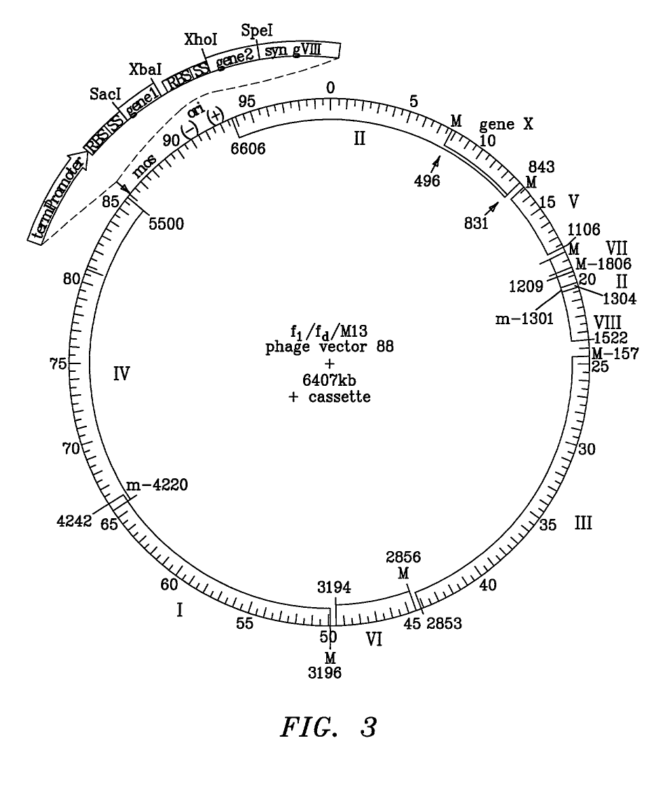

A novel vector was prepared by using the phage f1 genome as the starting material. A unique Nhe I restriction site was introduced into the f1 genome (GenBank accession #NC_{—}001397) between the end of gene IV and the MOS hairpin. (See FIG. 3) Overlap PCR was used to generate the restriction site between the end of gene IV and the MOS hairpin (see FIG. 3). The following pairs of primers were used to create the Nhe I site, which is underlined: 5′ CGCGCTTAATGCGCCGCTAGCTACAGGGCGCGTA 3′ (Seq. ID No. 1) was paired with primer 5′ GGTTAATTTGCGTGATGGACAGAC 3′ (Seq. ID No. 31) to generate a 213 bp fragment. 5′ TACGCGCCCTGTAGCTAGCGGCGCATTAAGCG 3′ (Seq. ID No. 32) was paired with primer 5′ GAAAAGCCCCAAAAACAGGAAGAT 3′ (Seq. ID No. 33) to generate a 502 bp fragment. The 213 bp fragment and the 502 bp fragments generated, which overlap, were then used in a PCR reaction to create a 681 bp fragment which contained two Psi I restriction sites that flanked the introduced Nhe I site. These Psi I sites were used to clone the final 492 bp fragment into Psi I digested F1 phage DNA. This causes a new NheI site to be created, which is underlined in the primer sequences. The double underline indicates the four additional bases generated by the creation of this restriction site. The incorporation of the new restriction site was verified using the resulting replicative form (RF) DNA of phage 205-13.1-1 by Nhe I digestion and/or sequence analysis. Additionally, the impact on phage assembly appeared minimal as plaque size of the wild type was similar to that of the modified phage. Plaque assays were performed by allowing dilutions of phage 205-13.1-1 to infect a bacterial host, then the mixture was plated in top agar onto an LB-agar plate. The plates were incubated overnight to allow a bacterial lawn to form. Circular areas of slower bacterial growth are the result of phage infection and were visualized on the plate. If the site of insertion/modification of the f1 genome interfered with the phage morphogenesis cycle, then the size of the clear circular plaque for the wild type would have been bigger and less turbid than that of the modified phage, but this was not the case.

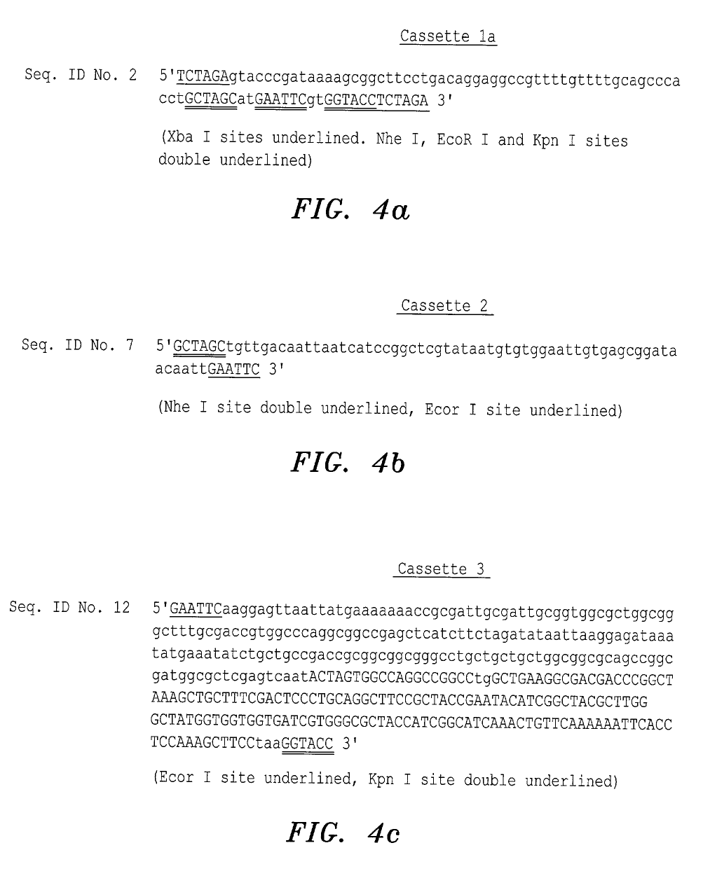

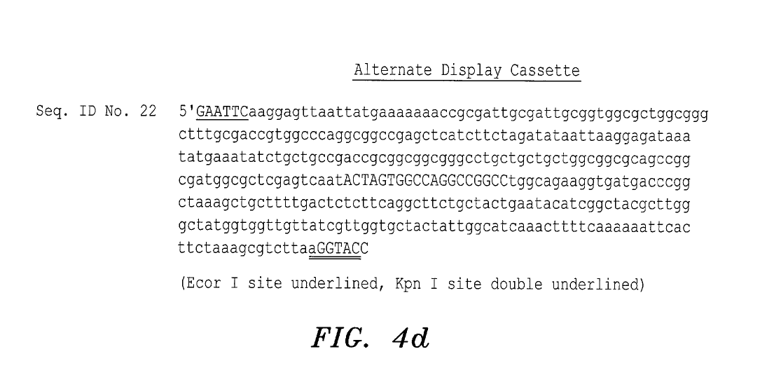

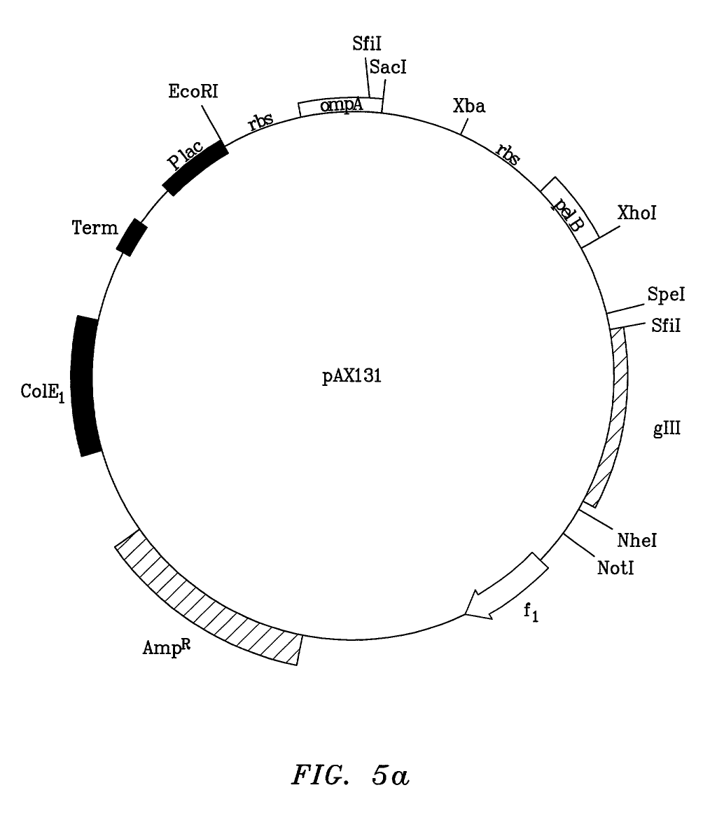

The modified f1 phage 205-13.1-1 was digested with the restriction endonuclease Nhe I and cassette 1a (Seq. ID No. 2) (see FIG. 4a), which contains a terminator (Krebber, A., Burmester, J., and Pluckthun, A., Gene (1996) 178, pp71-4) and multiple cloning sites was ligated into that position. Cassette 1 a was created by making use of long complimentary oligos which formed the double-stranded DNA cassette. The two oligos were mixed together at a 1:1 molar ratio, heat denatured and slowly cooled to allow the duplexed insert to form. The annealing of the oligos was such that single stranded DNA overhangs were present at each end (underlined). These overhangs are compatible with the Nhe I overhangs remaining after Nhe I digestion of the vector. However, the ends do not regenerate functional Nhe I sites after annealing. The oligos used for this construction method were:

| | |
|---|---|
| Cas. 1a-F2: | |
| 5′CTAGAGTACCCGATAAAAGCGGCTTCCTGACAGGAGGCCGTTTTGTTTTGCAGC | (Seq. ID No. 5) |
| CCACCTGCTAGCATGAATTCGTGGTACCT 3′ | |
| Cas. 1a-B2: | |
| 5′CTAGAGGTACCACGAATTCATGCTAGCAGGTGGGCTGCAAAACAAAACGGCCT | (Seq. ID No. 6) |
| CCTGTCAGGAAGCCGCTTTTATCGGGTACT 3′. | |

These two oligos contain Kpn I, Eco RI, and Nhe I sites (double underlined). As described above, the constructs were verified by both sequence analysis of the RF DNA and by analyzing plaque size, which remained unaltered. Insertion of this cassette generated phage 205-63.1.

The RF DNA of 205-63.1 was digested with Nhe I and Eco RI and cassette 2, (Seq. ID No. 7, see FIG. 4b) which contains the trc promotor, was added (see for example Invitrogen's pTrcHis A promotor sequence). Cassette 2 was generated by making use of long complementary oligos which formed the double stranded DNA cassette. The two oligos were mixed together at a 1:1 molar ratio, heat denatured and slowly cooled to allow the duplexed insert to form. The annealing of the oligos was such that single stranded DNA overhangs were present at each end, which were compatible with the NheI/EcoR I digested vector. The oligos used for construction were:

| | |
|---|---|
| Cas. 2-F2 | |
| 5′ CT AGC tgt tga caa tta atc atc cgg ctc gta taa tgt gtg | (Seq. ID No. 10) |
| gaa ttg tga gcg gat aac aat tG 3′ | |
| Cas. 2-B2 | |
| 5′ AAT TCa att gtt atc cgc tca caa ttc cac aca tta tac gag | (Seq. ID No. 11) |
| ccg gat gat taa ttg tca aca G 3′ | |

The Eco RI overhang is underlined, the Nhe I overhang is double underlined. Following verification of the resulting phage 205-87.4-3 by sequence analysis, a third cassette was inserted between the EcoRI and Kpn I sites of the modified fl. Cassette 3 (Seq. ID No. 12, see FIG. 4c) is the display cassette and contains the first and second cloning regions and a synthetic gene VIII from the vector pAX131-gene VIII (205-91, see below).

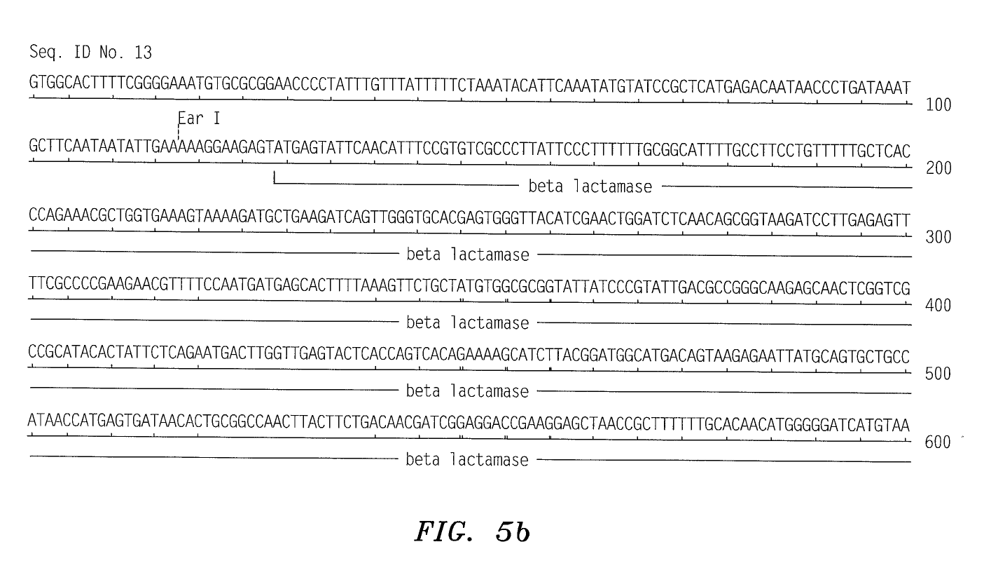

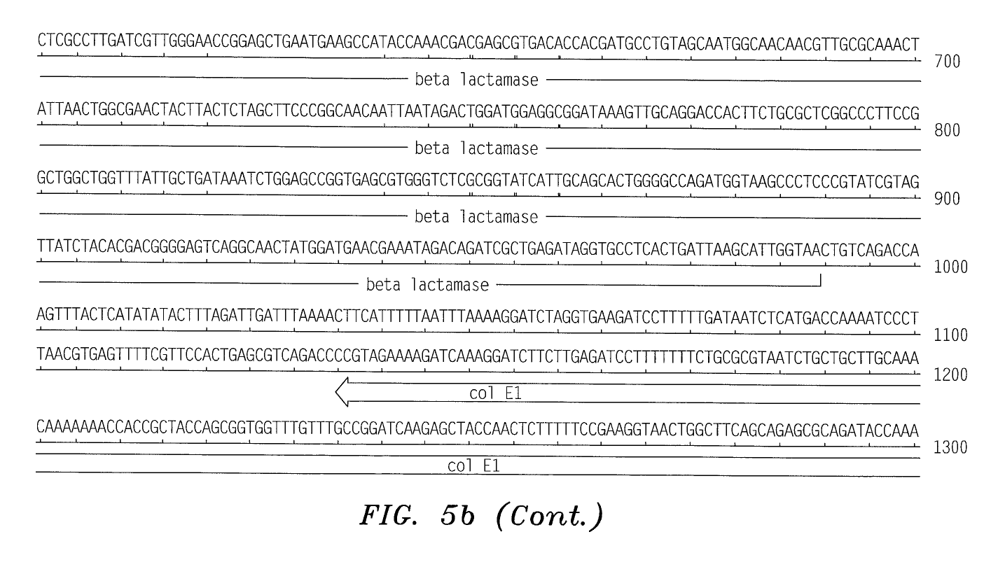

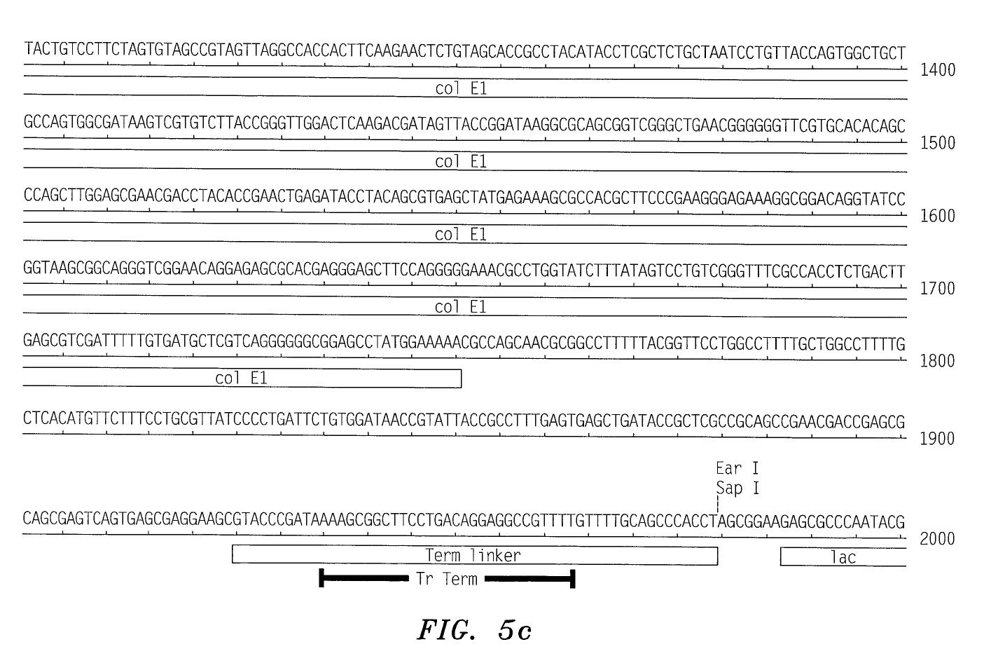

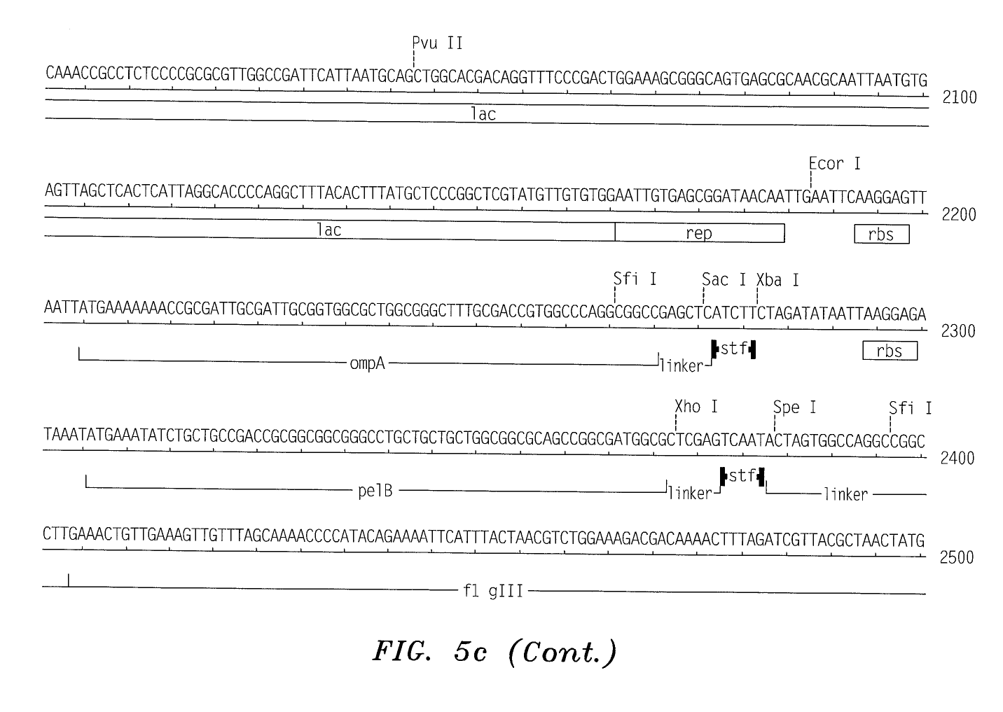

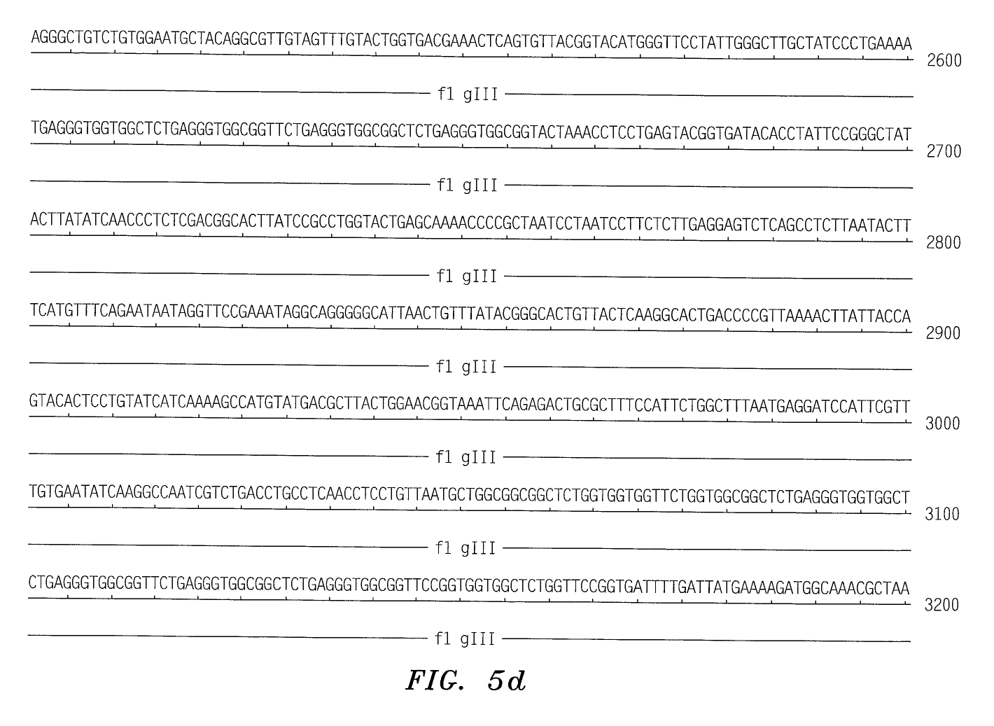

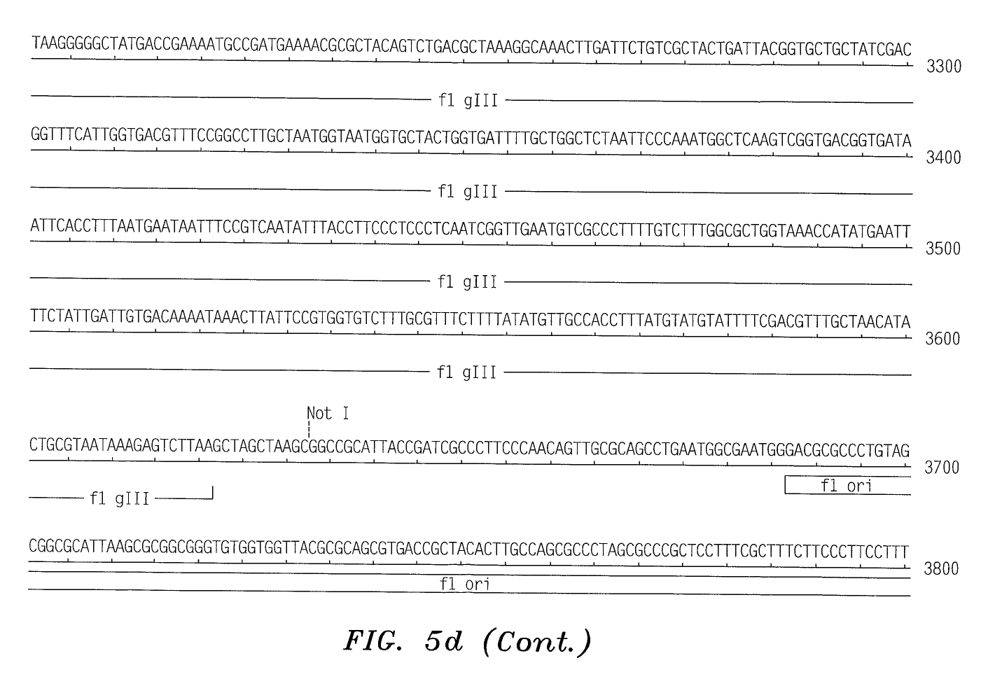

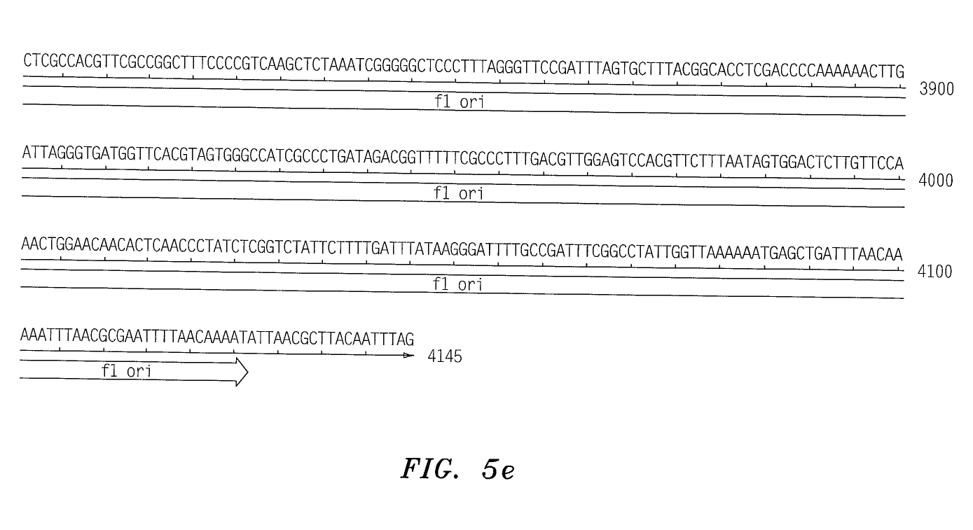

PAX131 is a phagemid vector prepared by modifying Bluescript II. FIG. 5a is a map of pAX131. FIGS. 5b-e show the nucleic acid sequence (Seq. ID No. 13) for pAX131. The preparation of pAX131 is described more fully in commonly owned pending application entitled PHAGEMID VECTORS filed on even date herewith under Express Mail Label No. EL820507456US (U.S. Provisional Application Serial No. 60/287,355, filed Apr. 27, 2001), the disclosure of which is incorporated herein in its entirety by this reference.

Preparation of Synthetic Gene VIII and the Display Cassette

The cloning region of pAX131 was constructed using an overlapping oligo approach (synthetically generated region). The area of interest includes a ribosomal binding site followed by an optimized (for E. coli expression) ompA leader sequence, an Sfi I, then Sac I and Xba I cloning sites for antibody light chains, another ribosomal binding sequence, an optimized pel B leader sequence, Xho I and Spe I heavy chain cloning sites followed by a downstream Sfi I. The portion of pAX131, which was replaced in the creation of cassette 3 includes the sequence for a gene III of f1 phage. See FIG. 5a.

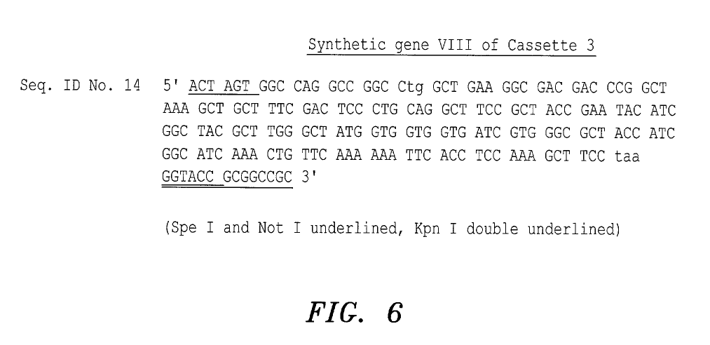

A synthetic gene VIII was generated with a nucleotide sequence optimized for bacterial expression (Seq. ID No. 14, see FIG. 6). The gene was assembled using overlapping phosphorylated oligonucleotides ligated together and cloned into a PCR script vector. The assembled gene was cut from this vector using the flanking Spe I and Not I sites and cloned into pAX131 at the same sites. The sequences of the overlapping oligos were:

| | |
|---|---|
| G8-1f: | |
| 5′CT AGT GGC CAG GCC GGC Ctg GCT GAA GGC GAC GAC CCG GCT AAA | (Seq. ID No. 15) |
| GCT GCT TTC GAC TCC CTG CAG GCT TCC GCT ACC GAA TAC ATC | |
| GGC TAC 3′ | |
| G8-2f: | |
| 5′GCT TGG GCT ATG GTG GTG GTG ATC GTG GGC GCT ACC ATC GGC ATC | (Seq. ID No. 16) |
| AAA CTG TTC AAA AAA TTC ACC TCC AAA GCT TCC taa GGT ACC GC 3′ | |
| G8-3b: | |
| 5′GGC CGC GGT ACC tta GGA AGC TTT GGA GGT GAA TTT TTT GAA CAG | (Seq. ID No. 17) |
| TTT GAT GCC GAT GGT 3′ | |
| G8-4b: | |
| 5′ AGC GCC CAC GAT CAC CAC CAC CAT AGC CCA AGC GTA GCC GAT | (Seq. ID No. 18) |
| GTA TTC GGT AGC GGA AGC CTG 3′ | |
| G8-5b: | |
| 5′ CAG GGA GTC GAA AGC AGC TTT AGC CGG GTC GTC GCC TTC AGC | (Seq. ID No. 19) |
| caG GCC GGC CTG GCC A 3′ | |

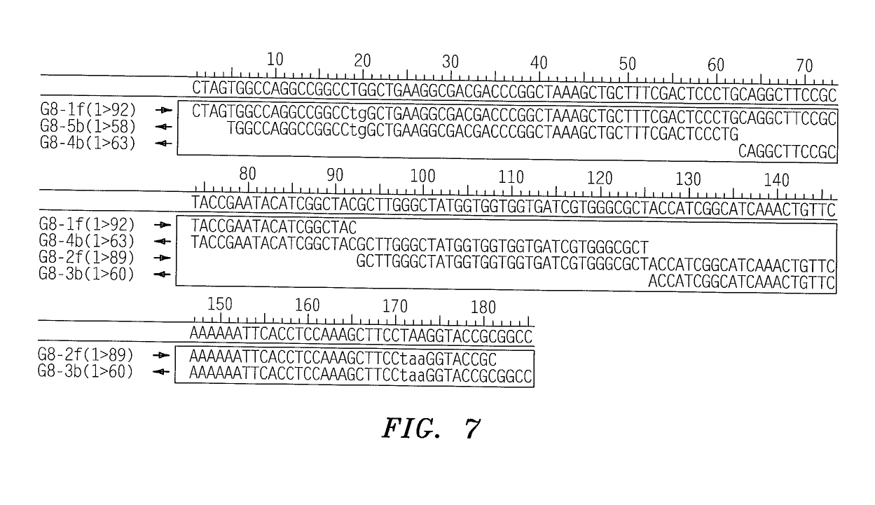

Spe I and Not I are shown underlined, and Kpn I is shown with a double underline. FIG. 7 shows the alignment of the oligos for the preparation of the synthetic gVIII. All oligo sequences are shown in the sense orientation, meaning reverse oligos G8-3b, G8-4b, and G8-5b are shown in FIG. 7 as their reverse complement in order to see the alignment with the forward oligos G8-1f and G8-2f. Construction of the synthetic gene was actually done by contract with Aptagen. The resulting vector 205-91 (see FIG. 8) was digested by restriction enzymes EcoR I and Kpn I to create the display cassette (Seq. ID No. 12, see FIG. 4c). This display cassette was then inserted into Eco RI and Kpn I digested 205-87 to create the final vector, 228-49.14.

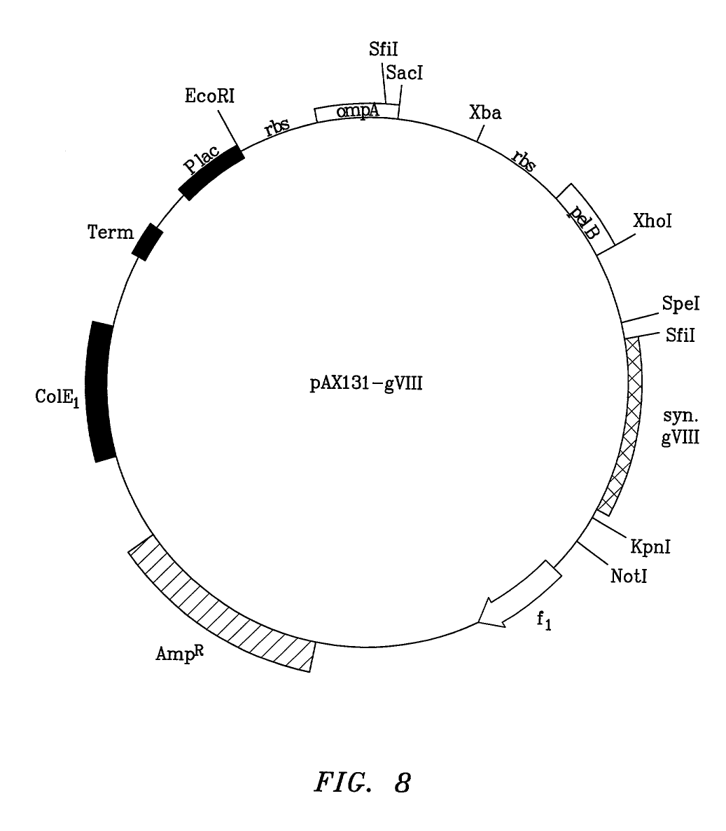

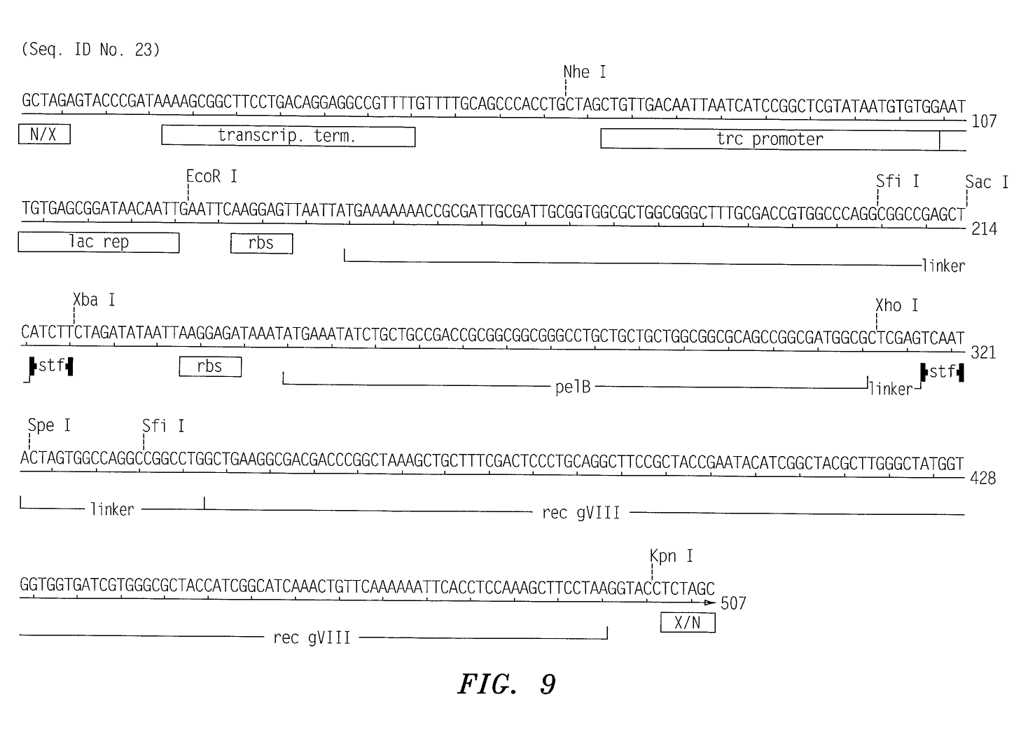

The sequence of the final inserted construct, 228-49.14, (Seq. ID. No. 23) resulting from the insertion of cassettes 1 a, 2 and 3 is shown in FIG. 9. Verification of the final construct includes sequence analysis of the resulting RF DNA and phage plaque size as described above. Additionally, a tetanus toxoid control antibody was cloned into the phage using the Sac I/Xba I sites for the light chain and Xho I/Spe I sites for the heavy chain Fd to create 241-15.29. Western blots of phage virion preps of 241-15.29 indicated that the Fab was expressed as a fusion protein with the synthetic gene VIII and incorporated into virions. A test panning experiment will also be performed to ensure that the Fab-fusion is presented on the phage surface and available for antigen selection. A phage mixture at a ratio of 1 specific phage/antibody into 10⁶ or more non-specific phage/antibody was used as the starting sample. Following 3 to 4 rounds of panning, the specific antibody was selected and therefore present at a much higher ratio than the starting ratio. Solid phase panning was also performed by adding 10¹⁰-10¹² phage to an antigen coated microtiter well for 1-2 hours at 37°. Non-specific phage were washed off with 0.5% Tween/PBS. Specific phage were eluted with low pH (such as 0.1M HCl, pH 2.2 with glycine) for 10 minutes at room temperature. Eluted phage were neutralized (with 2M Tris Base) and then added to bacterial cells to allow infection 15 minutes at room temperature. All cell/phage were plated in top agar on LB-agar plates and incubated overnight at 37°. The next day, phage were recovered from bacterial plaques by adding 5 mls media to each large petri dish and scrapping the top agar into 50 ml conical tubes. Agar debris was removed by centrifugation. Phage stock was used directly but can be concentrated by PEG precipitation if necessay: 4% PEG 8000+0.5M NaCl on ice for 30 minutes followed by centrifugation at 12,000×g for 20 minutes at 4°. The enriched phage were reselected in additional rounds of panning, typically 3-4 rounds total.

### EXAMPLE 2

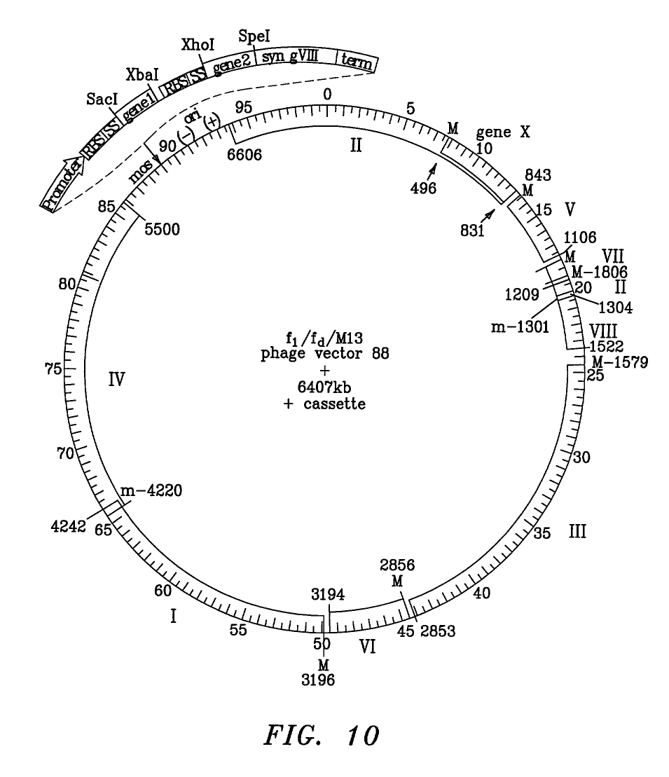

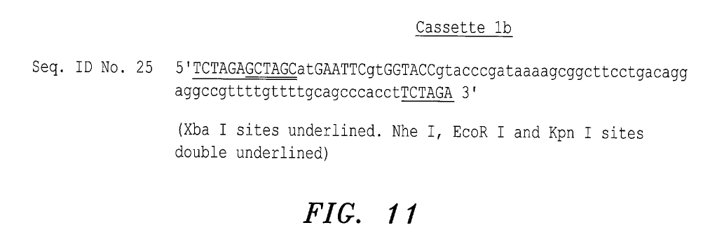

The overall scheme for modification of f1 at an alternate site is similar to that described above in Example 1. However, the insertion site for this Example is between the MOS hairpin and the minus origin. (See FIG. 10). As above, overlap PCR was used to generate the restriction site between the MOS hairpin and the minus origin (see FIG. 10). The following pairs of primers were used to create the Nhe I site, which is underlined: 5′ GAACGTGGCGAGAAAGCTAGCGAAGAAAGCGAAAGG 3′ (Seq. ID No. 24) was paired with primer 5′ GGTTAATTTGCGTGATGGACAGAC 3′ (Seq. ID No. 31) to generate a 305 bp fragment. 5′ CTTCGCTAGCTTTCTCGCCACGTTCGCC 3′ (Seq. ID No. 34) was paired with primer 5′ GAAAAGCCCCAAAAACAGGAAGAT 3′ (Seq. ID No. 33) to generate a 397 bp fragment. The 305 bp fragment and the 397 bp fragments generated, which overlap, were then used in a PCR reaction to create a 677 bp fragment which contained two Psi I restriction sites that flanked the introduced Nhe I site. These Psi I sites were used to clone the final 488 bp fragment into Psi I digested F1 phage DNA. The double underlines indicate the mutations introduced in order to create the Nhe I site. This procedure generated construct 205-13.2-1, which was sequenced for verification. Plaque size did not appear to be significantly altered by this mutation.

205-13.2-1 was digested with Nhe I. Cassette 1b was created for insertion into 205-13.2-1 by making use of long complementary oligos which form the double stranded DNA cassette. The two oligos were mixed together at a 1:1 molar ratio, heat denatured and slowly cooled to allow the duplexed insert to form. The annealing of the oligos was such that ends of single stranded DNA overhangs were present at each end, which were complementary to the Nhe I digested vector. Oligos used for this method were:

| | |
|---|---|
| Cas.1b-F2: | |
| 5′ CT AGA GCT AGC at GAA TTC gt GGT ACCgta ccc gat aaa agc ggc ttc ctg | (Seq. ID No. 28) |
| aca gga ggc cgt ttt gtt ttg cag ccc acc tT 3′ | |
| Cas.1b-B2: | |
| 5′ CTA GAa ggt ggg ctg caa aac aaa acg gcc tcc tgt cag gaa gcc gct ttt | (Seq. ID No. 29) |
| atc ggg tac GGT ACC ac GAA TTC at GCT AGC T 3′ | |

The overlapping ends are underlined, the Nhe I, Eco RI, and Kpn I sites are double underlined. The sequence is such that the ends do not regenerate a functional Nhe I site.

Insertion of Cassette 1b into 205-13.2-1 generated 205-83.1. This was digested with Nhe I and Eco RI and cassette 2, (Seq. ID No. 7, see FIG. 4b) was generated and added as described above in Example 1 to create construct 205-93.12. This was verified by sequence analysis, digested with Eco RI and Kpn I, and cassette 3 was inserted as described above in Example 1 to create the final construct 228-88.5. This was verified by sequence analysis and analysis of plaque size, which again did not appear to be significantly affected.

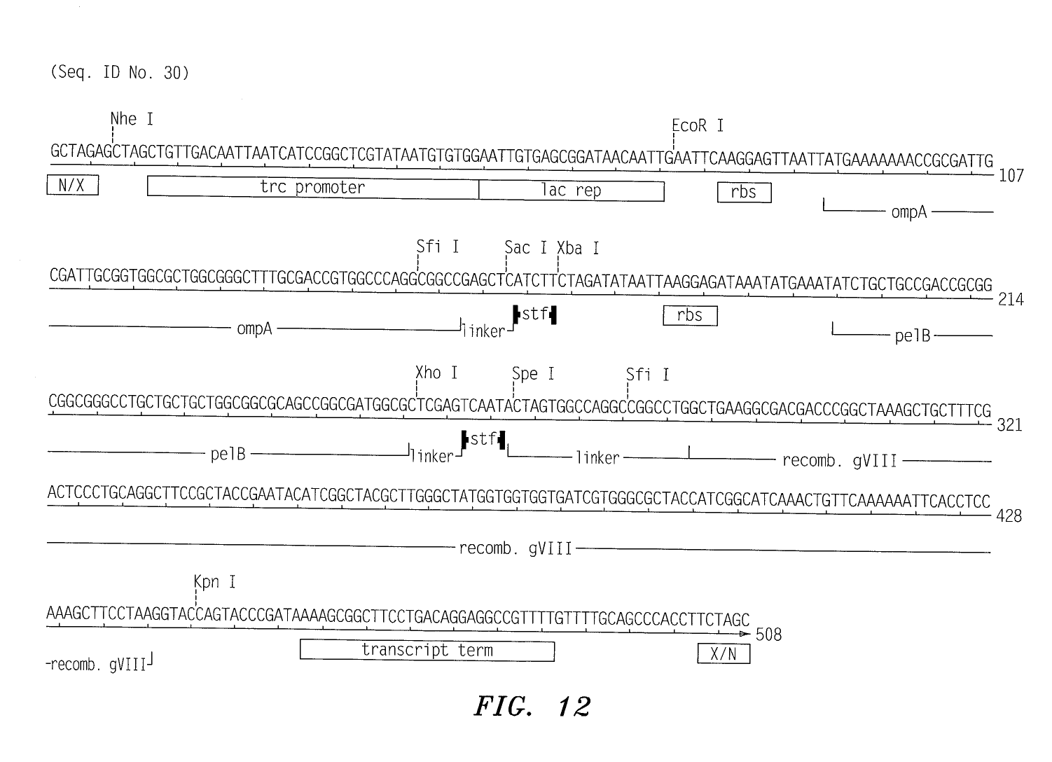

The sequence of the final inserted construct (Seq. ID No. 30) resulting from the insertion of cassettes 1b, 2 and 3 is presented in FIG. 12. Likewise a tetanus toxoid test antibody was inserted into the phage vector 228-88.5 to create phage 241-30.7 and this was analyzed for expression by Western Blot. This indicated that Fab attached to gene VIII expressed well and was incorporated into virions. A test panning experiment can also be performed as described above.

It is contemplated that the present novel vector can be used in connection with the production and screening of libraries made in accordance with conventional phage display technologies. Both natural and synthetic antibody repertoires have been generated as phage displayed libraries. Natural antibodies can be cloned from B-cell mRNA isolated from peripheral blood lymphocytes, bone marrow, spleen, or other lymphatic tissue of a human or non-human donor. Donors with an immune response to the antigen(s) of interest can be used to create immune antibody libraries. Alternatively, non-immune libraries may be generated from donors by isolating naïve antibody B cell genes. PCR using antibody specific primers on the 1^{st} strand cDNA allows the isolation of light chain and heavy chain antibody fragments which can then be cloned into the display vector.

Synthetic antibodies or antibody libraries can be made up in part or entirely with regions of synthetically derived sequence. Library diversity can be engineered within variable regions, particularly within CDRs, through the use of degenerate oligonucleotides. For example, a single Fab gene may be modified at the heavy chain CDR3 position to contain random nucleotide sequences. The random sequence can be introduced into the heavy chain gene using an oligonucleotide which contains the degenerate coding region in an overlap PCR approach. Alternatively, degenerate oligo cassettes can be cloned into restriction sites that flank the CDR(s) to create diversity. The resulting library generated by such approaches can then be cloned into a display vector in accordance with this disclosure.

Upon introduction of the display into bacteria, phage particles will be generated that have antibody displayed on the surface. The resulting collection of phage-displayed antibodies can be selected for those with the ability to bind to the antigen of interest using techniques known to those skilled in the art. Antibodies identified by this system can be used therapeutically, as diagnostic reagents, or as research tools.

It is contemplated that single and double stranded versions of the vectors described herein are within the scope of the present invention. It is well within the purview of those skilled in the art to prepare double stranded vectors from the single stranded nucleic acids described herein.

It will be understood that various modifications may be made to the embodiments described herein. For example, as those skilled in the art will appreciate, a first gene encoding a fusion protein having an antibody light chain to be fused to and displayed by pVIII and a second gene encoding a heavy chain Fd can be inserted into the vector at the newly created restriction site to provide effective antibody display. Therefore, the above description should not be construed as limiting, but merely as exemplifications of preferred embodiments. Those skilled in the art will envision other modifications within the scope and spirit of the claims appended hereto.

What is claimed:

1. A phage vector comprising:
a modified phage genome that contains, after gene IV but before the MOS hairpin:
a terminator;
a promoter;
a nucleotide sequence encoding at least a functional domain of pVIII; and
a first cloning site for receiving a first gene encoding a polypeptide to be displayed.

2. A phage vector as in claim 1 wherein the polypeptide to be displayed includes a heavy chain Fd.

3. A phage vector as in claim 1 wherein the polypeptide to be displayed includes a light chain.

4. A phage vector as in claim 1 further comprising a second cloning site positioned between the promoter and the first cloning site, the second cloning site being adapted to receive a second gene encoding a polypeptide capable of dimerizing to the polypeptide to be displayed.

5. A phage vector as in claim 4 wherein the second gene encodes an antibody light chain.

6. A phage vector as in claim 1 wherein the second gene encodes an antibody heavy chain Fd.

7. A phage vector as in claim 1 wherein the nucleotide sequence encoding at least a functional domain of pVIII encodes a truncated pVIII.

8. A phage vector comprising
a modified phage genome that contains, after the MOS hairpin but before the minus strand origin:
a promoter;
a cloning site for receiving a first gene encoding a polypeptide to be displayed;
a nucleotide sequence encoding at least a functional domain of pVIII; and
a terminator.

9. A phage vector as in claim 8 wherein the polypeptide to be displayed includes a heavy chain Fd.

10. A phage vector as in claim 8 wherein the polypeptide to be displayed includes a light chain.

11. A phage vector as in claim 8 further comprising a second cloning site positioned between the promoter and the first cloning site, the second cloning site being adapted to receive a second gene encoding a polypeptide capable of dimerizing to the polypeptide to be displayed.

12. A phage vector as in claim 11 wherein the second gene encodes an antibody light chain.

13. A phage vector as in claim 11 wherein the second gene encodes an antibody heavy chain Fd.

14. A phage vector as in claim 8 wherein the nucleotide sequence encoding at least a functional domain of pVIII encodes a truncated pVIII.

15. A method for producing a phage vector comprising:
incorporating a restriction site into a phage genome, the restriction site being located between gene IV and the MOS hairpin,
digesting at the incorporated restriction site; and
inserting a nucleotide sequence encoding at least a functional domain of pVIII and a first cloning site for receiving a first gene encoding a polypeptide to be displayed.

16. A method as in claim 15 wherein the first gene encodes an antibody heavy chain Fd.

17. A method as in claim 15 wherein the first gene encodes an antibody light chain.

18. A method as in claim 15 further comprising the step of inserting a second cloning site for receiving a second gene encoding a polypeptide capable of dimerizing to said polypeptide to be displayed.

19. A method as in claim 18 wherein the second gene encodes an antibody light chain.

20. A phage vector as in claim 18 wherein the second gene encodes an antibody heavy chain.

21. A method as in claim 15 wherein the nucleotide sequence encoding at least a functional domain of pVIII encodes a truncated pVIII.

22. A method for producing a phage vector comprising:
incorporating a restriction site into a phage genome, the restriction site being located between the MOS hairpin and the minus strand origin;
digesting at the incorporated restriction site; and
inserting a nucleotide sequence encoding at least a functional domain of pVIII and a first cloning site for receiving a first gene encoding a polypeptide to be displayed.

23. A method as in claim 22 wherein the first gene encodes an antibody heavy chain Fd.

24. A method as in claim 22 wherein the first gene encodes an antibody light chain.

25. A method as in claim 22 further comprising the step of inserting a second cloning site for receiving a second gene encoding a polypeptide capable of dimerizing to said polypeptide to be displayed.

26. A method as in claim 25 wherein the second gene encodes an antibody light chain.

27. A method as in claim 25 wherein the second gene encodes an antibody heavy chain.

28. A method as in claim 22 wherein the nucleotide sequence encoding at least a functional domain of pVIII encodes a truncated pVII.

29. A phage display library produced using the vector of claim 1.

30. A phage display library produced using the vector of claim 8.

31. A vector produced by the method of claim 15.

32. A vector produced using the method of claim 22.

33. A phage vector comprising
a phage genome modified to contain a restriction site after gene IV but before the MOS hairpin.

34. A phage vector as in claim 33 wherein the restriction site is selected from the group consisting of Nhe I, Hind III, Nco I, Xma I, Bgl II, Bst I and Pvu I.

35. A phage vector as in claim 33 wherein the restriction site is an Nhe I site.

36. A phage vector comprising
a phage genome modified to contain a restriction site after the MOS hairpin but before the minus strand origin.

37. A phage vector as in claim 36 wherein the restriction site is selected from the group consisting of Nhe I, Hind III, Nco I, Xma I, Bgl II, Bst I and Pvu I.

38. A phage vector as in claim 36 wherein the restriction site is an Nhe I site.
